(19) United States
(12) Patent Application Publication
(10) Pub. No.: US 2007/0293688 A1
(43) Pub. Date: Dec. 20, 2007

(54) Method for the Separation by Distillation of Pure Trioxane

(75) Inventors: Markus Siegert, Heidelberg (DE); Neven Lang, Mannheim (DE); Eckhard Stroefer, Mannheim (DE); Achim Stammer, Freinsheim (DE); Thorsten Friese, Mannheim (DE)

Correspondence Address: CONNOLLY BOVE LODGE & HUTZ, LLP, P O BOX 2207, WILMINGTON, DE, 19899, US

(73) Assignee: BASF Aktiengesellchaft, Ludwigshafen (DE)

(21) Appl. No.: 11/660,530

(22) PCT Filed: Aug. 18, 2005
(86) PCT No.: PCT/EP05/08944

§ 371 Date: Feb. 16, 2007

(30) Foreign Application Priority Data
Aug. 19, 2004 (DE) 10 2004 040 284.1

(51) Int. Cl. C07D 323/06

(52) U.S. Cl. 549/368

(57) ABSTRACT

The present invention relates to a process for distillatively removing pure trioxane from a feedstream (I) comprising trioxane in a proportion of at least 50% by weight, based on the total weight of the feedstream (I), and additionally formaldehyde and water, which comprises feeding the feedstream I and a further aqueous stream (II) which does not contain any components extraneous to the feedstream to a dividing wall column (TWK1) having a dividing wall TW which is arranged substantially perpendicularly and divides the column interior into a feed region (A1), a withdrawal reaction (B1), an upper combined column region (C1) and a lower combined column region (D1), and drawing off from the first dividing wall column (TWK1) a bottom stream (III) comprising pure trioxane and a sidestream (IV) at the withdrawal region (B1), comprising pure water.

The invention relates to a process for distillatively removing pure trioxane from a feed-stream comprising trioxane, formaldehyde and water.

Trioxane is prepared generally by reactive distillation of aqueous formaldehyde solution in the presence of acidic catalysts. The trioxane is subsequently extracted from the distillate comprising formaldehyde and water in addition to trioxane using halogenated hydrocarbons such as methylene chloride or 1,2-dichloroethane, or other water-immiscible solvents.

DE-A 1 668 867 describes a process for removing trioxane from mixtures comprising water, formaldehyde and trioxane by extraction with an organic solvent. In this process, an extraction section consisting of two subsections is charged at one end with a customary organic, virtually water-immiscible extractant for trioxane, and at the other end with water. Between the two subsections, the distillate of the trioxane synthesis to be separated is fed. On the side of the solvent feed, an aqueous formaldehyde solution is then obtained, and on the side of the water feed, a virtually formaldehyde-free solution of trioxane in the solvent. In one example, the distillate which is obtained in the trioxane synthesis and is composed of 40% by weight of water, 35% by weight of trioxane and 25% by weight of formaldehyde is metered into the middle section of a pulsation column, and methylene chloride is fed at the upper end of the column and water at the lower end of the column. In this case, an about 25% by weight solution of trioxane in methylene chloride is obtained at the lower end of the column and an about 30% by weight aqueous formaldehyde solution at the upper end of the column.

A disadvantage of this procedure is the occurrence of extractant which has to be purified. Some of the extractants used are hazardous substances (T or T⁺ substances in the context of the German Hazardous Substances Directive), whose handling entails special precautions.

DE-A 197 32 291 describes a process for removing trioxane from an aqueous mixture which consists substantially of trioxane, water and formaldehyde, by removing trioxane from the mixture by pervaporation and separating the trioxane-enriched permeate by rectification into trioxane and an azeotropic mixture of trioxane, water and formaldehyde. In the example, an aqueous mixture consisting of 40% by weight of trioxane, 40% by weight of water and 20% by weight of formaldehyde is separated in a first distillation column under atmospheric pressure into a water/formaldehyde mixture and into an azeotropic trioxane/water/formaldehyde mixture. The azeotropic mixture is passed into a pervaporation unit which comprises a membrane composed of polydimethylsiloxane with a hydrophobic zeolite. The trioxane-enriched mixture is separated in a second distillation column under atmospheric pressure into trioxane and, in turn, into an azeotropic mixture of trioxane, water and formaldehyde. This azeotropic mixture is recycled before the pervaporation stage.

A disadvantage of this procedure is the very high capital costs for the pervaporation unit.

The German patent application DE 103 61 516.4, unpublished at the priority date of the present application, discloses a process for distillatively removing trioxane from trioxane/formaldehyde/water mixtures which does not need extraction or pervaporation steps. However, the process requires a plant with three distillation columns for the removal of pure dioxane and pure water from the product mixture from a trioxane synthesis reactor.

It is accordingly an object of the invention to perform the same separation task, i.e. the removal of pure trioxane and of pure water from a trioxane/formaldehyde/water mixture with a small number of separation columns and accordingly with lower capital and operating costs.

The object is achieved by a process for distillatively removing pure trioxane from a feedstream containing trioxane in a proportion of at least 50% by weight, based on the total weight of the feedstream, and additionally formaldehyde and water, which comprises feeding the feedstream and a further aqueous stream which does not comprise any components extraneous to the feedstream to a first dividing wall column having a dividing wall which is arranged in the longitudinal direction of the column and divides the column interior into a feed region, a withdrawal region, an upper combined column region and a lower combined column region, and drawing off from the first dividing wall column a bottom stream comprising pure trioxane and a sidestream from the withdrawal region, comprising pure water.

It has been found that it is possible to separate a trioxane/formaldehyde/water feed mixture, provided that it comprises a relatively high proportion by weight of at least 50% by weight, preferably of from 60 to 80% by weight, of trioxane, in a single column to obtain pure trioxane and pure water.

In the present context, pure trioxane refers to a stream which comprises at least 97.5% by weight, preferably at least 99% by weight or 99.9% by weight or even 99.99% by weight, of trioxane, and pure water to a stream having a water content of at least 95.0% by weight, preferably of at least 99.0% by weight.

As most pure trioxane is defined a stream which comprises at least 99.95% by weight or 99.96% by weight or even 99.99% by weight trioxane.

By feeding a further aqueous stream not containing any components extraneous to the feed stream, it is possible to cross the limiting distillation line, running from the trioxane/water azeotrope on the binary side in the direction of the lowest-boiling ternary trioxane/formaldehyde/water azeotrope, in the direction of pure water.

In the inventive separation process, a dividing wall column is used, i.e. a distillation column having a dividing wall which is arranged in the longitudinal direction of the column, prevents mixing of liquid and vapor streams in subregions of the column and separates the column interior into a feed region, a withdrawal region, an upper combined column region and a lower combined column region.

Dividing wall columns are known and are described, for example, in EP-A 0 122 367, EP-A 0 126288 or EP-A 0 133510.

In an economically advantageous embodiment, the dividing wall is not welded into the column, but rather configured in the form of loosely inserted and adequately sealed subsegments.

The loose dividing wall advantageously has internal manholes or removable segments which allow access within the column from one side of the dividing wall to the other side thereof.

In one embodiment, the liquid distribution may deliberately be adjusted nonuniformly in the individual subregions of the first and/or second dividing wall column. In particular, the liquid may be introduced in the wall region of the dividing wall to an increased extent in the rectifying section of the feed region and/or of the withdrawal region, and to a reduced extent in the stripping section of the feed region and/or of the withdrawal region.

A trioxane/formaldehyde/water feedstream comprising at least 50% by weight of trioxane, based on the total weight of the feedstream, is introduced to the feed region of the first dividing wall column, preferably in the middle section thereof.

The feedstream preferably has the following composition: from 60 to 80% by weight of trioxane, from 10 to 30% by weight of water and from 3 to 20% by weight of formaldehyde, with or without additionally up to 15% by weight of low boilers selected from one or more of the substances listed below: methyl formate, methylal, dimethoxydimethyl ether, methanol, formic acid and further hemiacetals and full acetals.

Additionally fed to the first dividing wall column is a further aqueous stream which does not contain any components extraneous to the feedstream and whose water content is preferably at least 10% by weight, in particular at least 50% by weight.

Advantageously, the feedstream may be obtained by concentrating a crude trioxane stream which is obtained as a reactor effluent from a trioxane synthesis reactor by removal of low boilers and high boilers to a trioxane content of at least 50% by weight, preferably of at least 60% by weight, more preferably of at least 70% by weight.

The present process is not restricted with regard to the specific process procedure in the trioxane synthesis reactor. The crude trioxane stream obtained in the trioxane synthesis generally has the following composition: from 55 to 85% by weight of formaldehyde, from 15 to 35% by weight of water, from 1.0 to 30% by weight of trioxane and additionally low boilers and high boilers. In the present context, low boilers refer to substances whose boiling point is lower than the boiling point of pure trioxane, and high boilers to substances whose boiling point is higher than the boiling point of pure trioxane. In the present context, low boilers are in particular methylal, methanol and methyl formate, and high boilers are in particular dimethoxydimethyl ether and formic acid.

The crude trioxane stream is fed preferably to a dividing wall column in the feed region thereof, and the sidestream concentrated in trioxane is drawn off from the withdrawal region thereof and is conducted as a feed stream into the first dividing wall column.

In the second dividing wall column, low boilers are removed overhead and a stream containing high boilers is removed via the bottom and is preferably recycled into the trioxane synthesis reactor.

The bottom stream from the second dividing wall column comprises generally less than 1% by weight, preferably less than 0.1% by weight, of trioxane, more preferably less than 0.01% by weight of trioxane. The bottom stream has, for example, the following composition: from 65 to 85% by weight of formaldehyde, from 15 to 35% by weight of water and from 0 to 1% by weight of trioxane.

The second dividing wall column for the concentration of the crude trioxane stream is preferably operated at a top pressure in the range from 0.10 to 5.0 bar absolute, in particular at a top pressure in the range from 0.50 to 2.50 bar absolute.

The first dividing wall column, from which pure trioxane and pure water are removed, is advantageously operated at a higher top pressure than the second dividing wall column, specifically at a top pressure higher by from 0.1 to 15.0 bar than the top pressure of the second dividing wall column.

The first and/or the second dividing wall column are preferably designed in such a way that the number of theoretical plates is in each case between 4 and 90, preferably between 15 and 60.

In this case, the total number of theoretical plates in the feed region is preferably from 80 to 120%, more preferably from 90 to 100%, of the total number of plates in the withdrawal region of the first and/or of the second dividing wall column.

The theoretical plates in the first dividing wall column and/or in the second dividing wall column are divided between the individual column regions preferably as follows:
from 1 to 50%, preferably from 5 to 50%, of the total number of theoretical plates to the upper combined column region,
in each case from 1 to 75%, preferably from 5 to 50%, of the total number of theoretical plates to the rectifying section of the feed region and/or the stripping section of the feed region and/or the rectifying section of the withdrawal region and/or the stripping section of the withdrawal region and
from 1 to 50%, preferably from 5 to 50%, of the total number of theoretical plates to the lower combined column region. In the first and/or the second dividing wall column, the feed points for the particular feedstream and the withdrawal points for the particular side draw stream may be positioned preferably as follows:

The feed point for the feedstream into the feed region of the first dividing wall column and for the reactor effluent from a trioxane synthesis reactor into the feed region of the second dividing wall column are each arranged at a different height in the dividing wall column from the side draw point from the withdrawal region of the first dividing wall column and the side draw point from the withdrawal region of the second dividing wall column respectively, in particular separated by from 1 to 20, preferably by from 1 to 10, theoretical plates.

The feed region and/or the withdrawal region of the first dividing wall column and/or of the second dividing wall column are preferably provided fully or partly with structured packings or random packings. Advantageously, the dividing wall is designed with heat insulation in the regions provided with structured packings or random packings.

Both in the first and in the second dividing wall column, the side withdrawal stream may be drawn off either in liquid or gaseous-form.

The division of the vapor stream at the lower end of the dividing wall in the first and/or the second dividing wall column may be subject to a natural distribution.

In one process alternative, the vapor stream at the lower end of the dividing wall of the first and/or of the second dividing wall column, by virtue of the selection and/or dimensioning of the separating internals and/or by virtue of the incorporation of pressure drop-generating devices, in particular of diaphrams, may be adjusted in such a way that the ratio of the vapor stream in the feed region to the vapor stream in the withdrawal region is from 0.5 to 1.5, preferably from 0.9 to 1.1.

The effluent liquid from the upper combined column region of the first and/or of the second dividing wall column may preferably be collected in a collecting chamber disposed within or outside the dividing wall column and may preferably be divided by a fixed setting or control system at the upper end of the dividing wall in such a way that the ratio of the liquid stream to the feed region to the liquid stream to the withdrawal region is from 0.1 to 1.0, preferably from 0.25 to 0.8.

Advantageously, the liquid may be conveyed via a pump to the feed region or may be introduced under flow control using a static feed head of at least 1 m, preferably via closed-loop control in conjunction with the liquid level control of the collecting chamber, and the control system is adjusted such that the amount of liquid introduced to the feed region cannot fall below 30% of its normal value.

The amount of liquid withdrawn via the side draw of the withdrawal region may advantageously be controlled such that the amount of liquid introduced to the rectifying section of the withdrawal region cannot fall below 30% of its normal value.

In one embodiment, sampling means may be provided in the first and/or second dividing wall column at the upper and lower end of the dividing wall which enable samples in liquid or gaseous form to be taken continuously or at time intervals from the dividing wall column and to be analyzed with regard to their composition, preferably by gas chromatography.

The division ratio of the liquid at the upper end of the dividing wall in the first and/or second dividing wall column may advantageously be adjusted such that the concentration of those high-boiling components for which a certain limiting value for the concentration in the side draw should not be exceeded, in the liquid at the upper end of the dividing wall, is from 5 to 75%, preferably from 5 to 50%, of the limiting value in the side draw, and that the liquid division at the upper end of the dividing wall is adjusted to the effect that more liquid is passed to the feed region at higher contents of higher-boiling components, and less liquid at lower contents of higher-boiling components.

Advantageously, the concentration of low-boiling components for which a certain limiting value in the sidestream should not be exceeded, at the lower end of the dividing wall, is adjusted to from 10 to 99%, preferably from 25 to 97.5%, of the limiting value specified for the sidestream, and the heating output of the bottom evaporator is controlled to the effect that the heating output is increased at a higher content of low-boiling components and the heating output is reduced at a lower content of low-boiling components.

The top stream may be advantageously withdrawn from the first and/or second dividing wall column under temperature control and the measurement temperature used is a measurement point in the upper combined column region of the first and/or second dividing wall column which is disposed from 1 to 25, preferably from 1 to 10, theoretical plates below the upper end of the first and/or second dividing wall column.

The bottom product may advantageously be withdrawn from the first and/or second dividing wall column under temperature control and the control temperature used is a measurement point in the lower combined column region of the first and/or second dividing wall column which is disposed from 1 to 25, preferably from 2 to 15, theoretical plates above the lower end of the first and/or second dividing wall column.

The withdrawal of the sidestream from the withdrawal region of the first and/or second dividing wall column may preferably be under level control and the liquid level in the bottom evaporator may preferably be used.

Instead of the first and/or the second dividing wall column, an equivalent arrangement of two thermally coupled columns may be used, each of the thermally coupled columns preferably each being equipped with a dedicated evaporator and a dedicated condenser.

The thermally coupled columns may be operated at different pressures. Advantageously, only liquids are conveyed in the connecting streams between the two thermally coupled columns.

The bottom stream from the first of the thermally coupled columns may be evaporated partly or fully in an additional evaporator and subsequently fed to the second of the thermally coupled columns in biphasic form or in the form of a gaseous and of a liquid stream.

The feed stream may be pre-evaporated partly or fully and fed to the first dividing wall column or to the first of the thermally coupled columns in biphasic form or in the form of a gaseous and of a liquid stream.

In a further preferred embodiment only one single dividing wall column is used, which corresponds to the previously mentioned first dividing wall column and to which accordingly the previously mentioned feed stream is fed, preferably in the middle part thereof, and which contains at least 50% by weight trioxane, related to the total weight of the feed stream.

In addition to what is afore mentioned for obtaining this feed stream for the dividing wall column, the process variants which are described in the following are also possible:

The reactor withdrawal from the trioxane synthesis reactor is fed to a first distillation column with at least 2, preferably 2 to 50 theoretical plates, which is operated at a top pressure between 0.1 and 2 bar absolute, preferably 0.5 to 2 bar absolute, e.g. 1 bar absolute.

The stripping section includes in general at least 25% of the total number of the theoretical plates of the column, preferably 50 to 90%. The feed stream to the first distillation column, which is the reactor withdrawal from the front end dioxane-synthesis reactor, comprises in general 35 to 80% formaldehyde, 25 to 45% water and 1 to 30% by weight trioxane. This mixture is separated in the first distillation column in a stream from the lower section of the first distillation column, especially a bottom stream and a stream from the upper section of the first distillation column, especially a top stream. The stream from the lower section of the first distillation column comprises in general 51 to 80% by weight formaldehyde, 20 to 49% by weight water and 0 to 1% by weight trioxane and is preferably recycled into the trioxane-synthesis reactor. The stream from the upper part of the distillation column comprises in general 1 to 15% by weight of formaldehyde, 15 to 35% by weight of water and 60 to 80% by weight of trioxane and is fed to a second distillation column for separating low boilers.

The trioxane-synthesis reactor can also be combined with the first distillation column in a reactive distillation column. This reactive distillation column can include in the stripping section a catalyst fixed bed of a heterogeneous catalyst. Alternatively, the reactive distillation can also be processed in the presence of a homogeneous catalyst.

The top stream from the first distillation column is preferably fed to a second distillation column for separating low boilers. Usual low boilers which can be formed in the trioxane synthesis and in the distillative separation are: methyl formate, methylal, dimethoxydimethyl ether, trimethoxydimethylether, methanol, formic acid as well as further hemiacetals and full acetals. The low boilers are separated preferably via top of the second distillation column, which is preferably processed at a pressure from 1 to 2 bar. In general, the column for separating low boilers has at least 5 theoretical plates, preferably 15 to 50 theoretical plates. Preferably, the stripping section of this column comprises 25 to 90% of the theoretical plates of this column.

For specially narrow specification requests, that it is for obtaining trioxane with a purity degree corresponding to the previously defined minimum content for most pure trioxane it is possible to feed the pure trioxane stream to a further third distillation column, which has the function of a most pure column and wherein heavy boilers with respect to trioxane are separated. The trioxane most pure column can comprise especially between 5 and 20 theoretical plates and be processed at atmospheric pressure and also at lower or higher pressure with respect to atmospheric pressure.

With respect to separating internals which may be used there are no limitations.

The trioxane most pure column is equipped especially with stripping and washing section, but it may be also a pure stripping column without washing section. From the trioxane most pure column in the upper section thereof, preferably from top of the column, a stream containing most pure trioxane is withdrawn, which is condensed in a condenser on top of the column, partially returned as reflux into the column and the remaining being withdrawn as value product stream. The bottom stream from the trioxane most pure column, which still contains heavy boilers with respect to trioxane, is preferably recycled into the trioxane synthesis reactor.

The pure or most pure trioxane obtained is preferably used to prepare polyoxymethylene, polyoxymethylene derivatives such as polyoxymethylene dimethyl ether, and diaminodiphenylmethane.

The invention is illustrated in detail hereinbelow with reference to a drawing and an example:

The figures show:

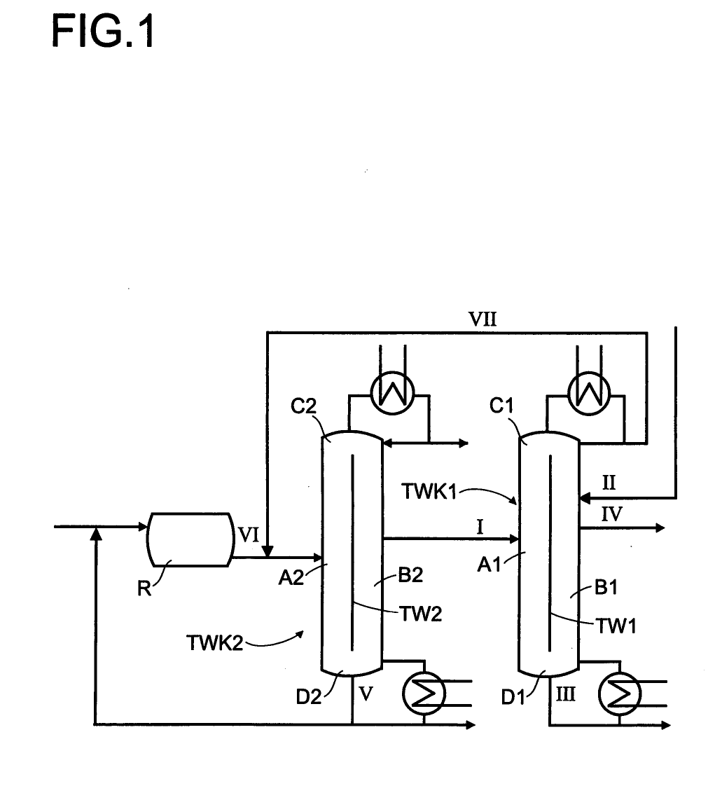

FIG. 1 the schematic illustration of a plant for carrying out a preferred embodiment of the process according to the invention and

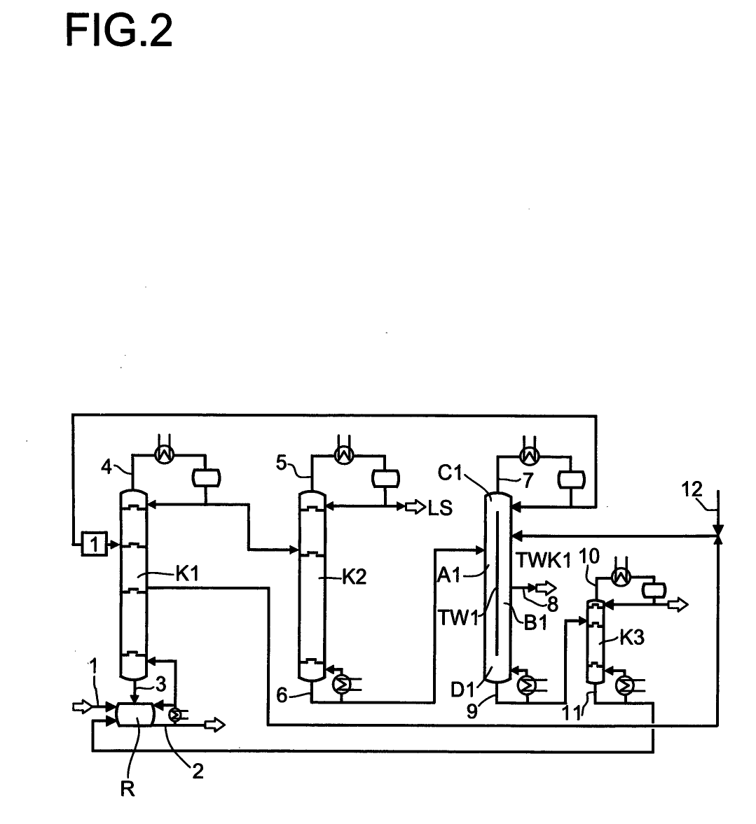

FIG. 2 the schematic illustration of a plant for carrying out a further preferred embodiment of the process according to the invention.

The plant shown in FIG. 1 has two dividing wall columns, TWK 1 and TWK2, each having a dividing wall arranged in the longitudinal direction of the column, TW 1 and TW 2, which divide the column interior in each case into a feed region, A1, A2, a withdrawal region B1, B2, an upper combined column region C1, C2, and a lower combined column region D1, D2. The dividing wall columns TWK1 and TWK2 each have bottom evaporators and condensers at the top of the column. Connected upstream of the second dividing wall column TWK 2 is a trioxane synthesis reactor R.

A formaldehyde-rich aqueous solution is fed to the trioxane synthesis reactor which is configured as an evaporator, stirred vessel, fixed bed reactor or fluidized bed reactor. From the trioxane synthesis reactor, a trioxane/formaldehyde/water mixture VI is drawn off, combined with the recycle stream VII which is obtained as a top stream from the first dividing wall column TWK1 and introduced to the feed region A2 of the second dividing wall column TWK2. In the second dividing wall column TWK2, a formaldehyde-rich bottom draw stream V is obtained which is recycled into the trioxane synthesis reactor R, and also a sidestream which is introduced as the feedstream I to the feed region A1 of the first dividing wall column TWK1.

A further aqueous stream II is additionally fed to the first dividing wall column TWK1 at a suitable point thereof.

From the first dividing wall column TWK1, a bottom stream III comprising pure trioxane and a side draw stream IV comprising pure water are drawn off.

According to the specially preferred process variant represented in FIG. 2, an aqueous, formaldehyde rich stream 1, with a formaldehyde content of usually 50 to 80% by weight, is fed to the trioxane synthesis reactor R, which is an evaporator, a stirring vessel, a fixed or a flowing bed reactor. The trioxane/formaldehyde/water-mixture 2 leaving the trioxane synthesis reactor R is fed to the first distillation column K1 and separated therein into a bottom stream 3, containing formaldehyde and water, and a top stream 4, containing formaldehyde, water and trioxane. The bottom stream 3 is recycled into the trioxane synthesis reactor R.

The top stream 4 is condensed in a condenser on top of the column and partly returned as reflux on the column R1 and the remainder fed to a second column K2 for separating low boilers. From the column K2 a top stream 5, containing low boilers, i.a. methyl formate, methylal, dimethoxydimethylether and methanol is withdrawn and condensed in a condenser on top of the column, partly returned as reflux on the column and the remainder drawn off. The bottom stream 6 from the low boilers separating column K2 is fed to the first dividing wall column TWK1 which is constructed as described with reference to FIG. 1 and from which a top stream 7 is withdrawn, which is condensed in a condenser on the top of the column, partly returned as reflux on the first separating wall column TWK1 and the remainder recycled into the first distillation column A1. From the withdrawal region B1 of the separating wall column TWK1 a side stream 8 is withdrawn, corresponding to the side stream IV of the process variant represented in FIG. 1 as well as a bottom stream 9, containing pure trioxane, which corresponds to the bottom stream III of the process variant represented in FIG. 1. The bottom stream 9 from the dividing wall column TWK1 is fed to a third distillation column K3 and separated therein into a top stream 10, containing most pure trioxane, and a bottom stream 11, which is recycled into the trioxane synthesis reactor.

A water rich stream 12 is fed to the separating wall column TWK1 and to the distillation column K1, in each case at a suitable position thereof.

1-52. (canceled)

53. A process comprising:
(a) providing a feedstream and a fit aqueous steam to a first dividing wall column, wherein the feedstream comprises trioxane, formaldehyde and water, the trioxane being present in the feedstream in an amount of at least 50% by weight based on the total weight of the feedstream, wherein the further aqueous stream contains no components extraneous to the feedstream, and wherein the first dividing wall column has a dividing wall arranged within an interior of the column in a longitudinal direction of the column and wherein the dividing wall divides the interior of the column into a feed region, a withdrawal region, an upper combined column region and a lower combined column region;
(b) distilling the feedstream and other aqueous stream within the column to separate the trioxane, water and formaldehyde; and
(ac) removing; (i) from the lower combined column region a bottom stream comprising pure trioxane, and (ii) from the withdrawal region a sidestream comprising pure water.

54. The process according to claim 53, wherein the feedstream comprises 60 to 80% by weight of trioxane, 10 to 30% by weight of water and 3 to 20% by weight of formaldehyde.

55. The process according to claim 53, wherein the first aqueous stream comprises at least 10% by weight of water.

56. The process according to claim 53, wherein the feedstream comprises a trioxane synthesis reactor effluent concentrated to a trioxane content of at least 50% by weight by removing low boilers and high boilers.

57. The process according to claim 56, wherein the feedstream comprises a side draw stream from a second dividing wall column,

58. The process according to claim 57, wherein a bottom stream from the second dividing wall column, comprising high boilers, is recycled into the trioxane synthesis reactor.

59. The process according to claim 57, wherein the second dividing wall column has a top pressure of 0.10 to 5.0 bar absolute.

60. The process according to claim 59, wherein the first dividing wall column has a top pressure of 0.1 to 15.0 bar higher than the top pressure of the second dividing wall column.

61. The process according to claim 53, wherein one or both of the first dividing wall column and the second dividing wall column has a number of theoretical plates of 4 to 90.

62. The process according to claim 61, wherein 1 to 50% of the total number of theoretical plates in one or both of the first dividing wall column and the second dividing wall column are in the upper combined column region, wherein in each case from 1 to 75% of the total number of theoretical plates in one or both of the first dividing wall column and the second dividing wall column are in the feed region and/or the withdrawal region; and wherein 1 to 50% of the total number of theoretical plates in one or both of the first dividing wall column and the second dividing wall column are in the lower combined column region.

63. The process according to claim 53, wherein the feedstream is fed into the feed region of the first dividing wall column at a feed point arranged at a different height in the dividing wall column than a side draw point in the withdrawal region of the first dividing wall column for removal of the sidestream.

64. The process according to claim 53, wherein one or both of the feed region and the withdrawal region of the first dividing wall is at least partially provided with structured or random packings and the dividing wall is designed with heat insulation in regions provided with structured or random packings.

65. The process according to claim 53, wherein during distilling vapors at a lower end of the dividing wall of the first dividing wall column are divided such that the ratio of the vapors in the feed region to vapors in the withdrawal region is 0.5 to 1.5.

66. The process according to claims 53, wherein an effluent liquid from the upper combined column region of the first dividing wall column is collected in a collecting chamber disposed within or outside the dividing wall column and is divided and returned to the feed region and the withdrawal region by a fixed setting or control system at an upper end of the dividing wall such that the ratio of the effluent liquid to the feed region to the effluent liquid to the withdrawal region is 0.1 to 1.0.

67. The process according to claim 66, wherein the effluent liquid is conveyed via a pump to the feed region and is introduced under flow control via a static feed head of at least 1 m and wherein the control system is adjusted such that the amount of effluent liquid introduced to the feed region does not fall below 30% of its normal value.

68. The process according to claim 53, wherein the amount of sidestream removed from the withdrawal region is controlled such that the amount feedstream and Her aqueous stream in the withdrawal region does not fall below 30% of its normal value,

69. The process according to claim 53, wherein the first dividing wall column farther comprises sample access at an upper and a lower end of the dividing wall, and wherein the process former comprises taking samples in liquid or gaseous form from the dividing wall column, and analyzing the samples for composition.

70. The process according to claim 53, wherein the division ratio of liquid at an upper end of the dividing wall is adjusted such that the concentration of high-boiling components for which a certain limiting value for the concentration in the side draw should not be exceeded, in the liquid at the upper end of the dividing wall, is from 5 to 75% of the limiting value in the side draw, and that the liquid division at the upper end of the dividing wall is adjusted such that more liquid is passed to the feed region at higher contents of higher-boiling components, and less liquid at lower contents of higher-boiling components,

71. The process according to claim 53, wherein the concentration of low-boiling components for which a certain limiting value in the sidestream should not be exceeded, at a lower end of the dividing wall, is adjusted to from 10 to 99% of the limiting value specified for the sidestream, and heating output of a bottom evaporator is adjusted such that the heating output is increased at a higher content of low-boiling components and the heating output is reduced at a lower content of low-boiling components.

72. The process according to claim 53, wherein a top stream is withdrawn from the dividing wall column under temperature control, wherein a measurement point in the upper combined column region of the dividing wall column which is disposed from 1 to 25 theoretical plates below an upper end of the dividing wall column is used to define a control temperature.

73. The process according to claim 53, wherein the bottom stream is removed under temperature control, wherein a measurement point in the lower combined column region of the dividing wall column which is disposed from 1 to 25 theoretical plates above a lower end of the dividing wall column is used to define a control temperature.

74. The process according to claim 53, wherein the removal of the sidestream from the withdrawal region of the dividing wall column is under level control and a liquid level in a bottom evaporator is used as a control parameter.

75. The process according to claim 53, wherein the first dividing wall column is substituted with a connection of two thermally coupled columns, each of the thermally coupled columns having a dedicated evaporator and a dedicated condenser.

76. The process according to claim 75, wherein the thermally coupled columns are operated at different pressures and only liquids are conveyed in connecting streams between the two thermally coupled columns.

77. The process according to claim 75, wherein a bottom stream from the first of the thermally coupled columns is at lest partly evaporated in an additional evaporator and is subsequently fed to the second of the thermally coupled columns in biphasic form or in the form of a gaseous and of a liquid stream.

78. The process according to claim 53, wherein the feedstream is at least partly pre evaporated and is fed to the first dividing wall column in biphasic form or in the form of a gaseous and of a liquid stream.
